(19) United States
(12) Patent Application Publication
(10) Pub. No.: US 2024/0161903 A1
(43) Pub. Date: May 16, 2024

(54) DATA COMMUNICATION BETWEEN A VENTILATOR AND AT LEAST ONE DATA PROCESSING DEVICE

(71) Applicant: Loewenstein Medical Technology S.A., Luxembourg (LU)

(72) Inventor: Matthias SCHWAIBOLD, Karlsruhe (DE)

(21) Appl. No.: 18/500,240

(22) Filed: Nov. 2, 2023

(30) Foreign Application Priority Data
Nov. 11, 2022 (DE) 102022129950.3

(51) Int. Cl. G16H 20/40; A61M 16/00

(52) U.S. Cl. CPC G16H 20/40; A61M 16/022; A61M 2205/3334; A61M 2205/3344; A61M 2205/502; A61M 2230/42

(57) ABSTRACT

A transmitting module comprises elements for performing the following: receiving input data comprising sensor data defining at least one parameter which is detected by at least one sensor and is relevant to ventilation of a patient by a ventilator and/or comprising user data encoding information regarding a patient who is ventilated or to be ventilated by a ventilator, said information being input via a user interface; evaluating the input data to recognize an event from among a plurality of possible events, each possible event being relevant to ventilation of the patient by the ventilator; transmitting a message regarding the recognized event to a receiving module via a data communication connection connecting the ventilator to a data processing device situated outside the ventilator for data communication.

### CROSS-REFERENCE TO RELATED APPLICATIONS

The present application claims priority under 35 U.S.C. § 119 of German Patent Application No. 10 2022 129 950.3, filed Nov. 11, 2022, the entire disclosure of which is expressly incorporated by reference herein.

### BACKGROUND OF THE INVENTION

### 1. Field of the Invention

The invention relates to a transmitting module for transmitting a message via a data communication connection between a ventilator and at least one data processing device situated outside the ventilator. Furthermore, the invention relates to a receiving module for receiving a message via such a data communication connection, a corresponding transmitting and respectively receiving method and also a transmitting and receiving system, a computer program and a computer-readable medium for performing the method. In addition, the invention relates to a ventilator and a ventilation system.

### 2. Discussion of Background Information

Modern ventilators such as home ventilators, sleep respiratory therapy apparatuses, intensive care ventilators or anaesthesia apparatuses may be networked with a central data processing device, for example a (cloud) server. This enables central monitoring of the sensor data provided by a ventilator. Further clients may additionally be networked with the server and may transmit requests to the server and/or receive them from the server and/or may communicate with the ventilator via the server, for instance in order to carry out remote maintenance or to set ventilation parameters remotely. In this case, depending on the number of network subscribers and depending on the type of data transferred, considerable amounts of data may arise and may greatly load the data communication network or require very high-performance and correspondingly expensive data communication connections.

In view of the foregoing, it would be advantageous to be able to improve the transfer of data between a ventilator and one or more data processing devices in a data communication network.

### SUMMARY OF THE INVENTION

A first aspect of the invention relates to a transmitting module. The transmitting module comprises means for performing at least the following steps: receiving input data, comprising sensor data and/or user data, wherein the sensor data define at least one parameter which is detected by at least one sensor and is relevant to ventilation of a patient by a ventilator, and the user data encode information regarding a patient who is ventilated or to be ventilated by a ventilator, said information being input by a user via a user interface; evaluating the input data in order to recognize an event from among a plurality of possible events, wherein each possible event is relevant to ventilation of the patient by the ventilator; transmitting a message regarding the recognized event to a receiving module via a data communication connection connecting the ventilator to at least one data processing device situated outside the ventilator for data communication purposes.

To put it another way, the approach presented here enables event-controlled data communication between the ventilator and a data processing device (or a plurality of data processing devices) by virtue of data in the form of messages being transferred only if a specific event relevant to ventilation is recognized. This has the effect that the amount of data transferred is significantly reduced in comparison with embodiments in which messages are transferred at specific time intervals independently of the message content, which improves the efficiency of the data communication and prevents overloading of the data communication network. A further advantage is that fewer costs are incurred overall during the data communication since more cost-effective data tariffs and/or lower-performance hardware components can be used without crucial functional losses or information losses. Particularly in very large data communication networks, for example within a large clinic or if hundreds or even thousands of (home) ventilators from different locations communicate with a central server for monitoring and/or maintenance purposes, the reduction of the (payload) data to be transferred such as is made possible by the approach presented here can have a considerable influence on costs.

Certain terms are explained in greater detail hereinafter.

“Module” as in “transmitting module” or “receiving module” can be understood to mean a software and/or hardware module. Such a module can be a component of the ventilator and/or of the data processing device. By way of example, the transmitting or receiving module can be embodied as a software module of specific control and/or monitoring software and/or of a specific app in the ventilator or in the data processing device. Alternatively, the transmitting or receiving module can be a hardware module having a processor, wherein the processor can be configured to execute a specific computer program (see further below).

“Ventilator” can be understood to mean for example a home ventilator, a sleep respiratory therapy apparatus, an intensive care ventilator, an anaesthesia apparatus or a combination of at least two of these examples. The ventilator can be suitable for invasive and/or non-invasive ventilation of the patient. The ventilator can be portable for carrying by the patient, or stationary.

“Data processing device” can be understood to mean generally a device comprising a computer. Accordingly, the data processing device can comprise a processor, a memory and data communication interfaces for wireless and/or wired data communication with peripherals, i.e., the ventilator and/or other data processing devices. The data processing device can be for example a (cloud) server a PC, a laptop, a tablet, a smartphone, a smartwatch or a combination of at least two of these examples. However, the data processing device can also be a further ventilator.

“Sensor” can be understood to mean a component of the ventilator itself or a sensor which is separate from the ventilator, for example carried by the patient. The sensor data may have been provided in particular using different types of sensors. Such a sensor can be embodied for example as pressure sensor, volumetric flow sensor, moisture sensor, pulse oximeter or thermometer. A respiratory gas volumetric flow (also called flow) can be determined for example by measurement of a differential pressure, by thermal anemometry, by means of an ultrasound method or on the basis of a combination of at least two of these examples. By way of example, infrared-optical, spectroscopic, electromagnetic or electrochemical methods or a plurality of these methods in combination can be used for the measurement of a respiratory gas composition.

“User” can be understood to mean the patient themselves or a person other than the patient, for example an operator of the ventilator or a physician or a caregiver of the patient.

“User data” can be understood to mean generally data regarding a state of health of the patient. Specifically, the user data can provide for example information about a course of therapy, compliance, a diagnosis, a medical history and/or medication of the patient. To put it another way, the user data can encode results of one or more medical examinations of the patient and/or answers of the patient (or generally of an operator of the ventilator) to specific questions relevant to ventilation. Such questions may have been displayed and/or answered for example, via the user interface (e.g., in a corresponding app).

The input data can additionally comprise log data or fault messages of the ventilator.

“User interface” can be understood to mean generally a human-machine interface. By way of example, the user interface can comprise a user interface—displayed on a (touch) display—of a specific app that runs on the ventilator and/or the data processing device (networked therewith), and/or separate operating elements such as a keyboard or a mouse. The user interface can be a hardware and/or software component of the ventilator and/or of the at least one data processing device. However, the user interface can also be a hardware and/or software component of an (additional) user device connected to the ventilator and/or the at least one data processing device for data communication purposes. Such a user device can be for example a PC, a laptop, a tablet or a smartphone of the user. In this case, therefore, the user device functions as the user interface of the ventilator and/or of the at least one data processing device.

“Event” can be understood to mean a medical and/or technical event. By way of example, the recognized event can be a medical and/or technical complication which may increase the risk of a significant deterioration in the patient's state of health, in particular the patient's ability to breathe. However, a significant improvement in the patient's state of health, in particular the patient's ability to breathe, can also be recognized as an event (examples of possible events are described in greater detail further below).

The data communication connection can enable wireless and/or wired data communication in a LAN, WAN and/or GAN. By way of example, the data communication connection can be at least one of the following connections: an Internet connection, a WLAN connection, a mobile radio connection, a Bluetooth connection.

The data communication connection can connect the ventilator and the data processing device(s) to one another indirectly, i.e., by means of at least one further data processing device, and/or directly, i.e., without a detour via one or more further data processing devices.

By way of example, the data communication connection can connect the ventilator to a (mobile) terminal as the data processing device (e.g., a PC, a laptop, a tablet or a smartphone) via a server, i.e., indirectly. However, a direct data communication connection between the ventilator and the terminal is also possible.

A further possibility is for the data communication connection to connect the ventilator to a server as the data processing device via a (mobile) terminal (e.g., a PC, a laptop, a tablet or a smartphone). By way of example, the ventilator, preferably via Bluetooth and/or WLAN, can be coupled to the terminal in order to exchange data with the terminal, wherein the terminal, preferably via the Internet, can be coupled to the server in order to exchange data with the server. However, a direct data communication connection between the ventilator and the server is also possible.

The input data can be received at least in part via the data communication connection in the transmitting module. By way of example, at least one portion of the input data, i.e., at least one portion of the sensor data and/or of the user data, can be received in the data processing device from the ventilator (or vice versa).

A second aspect of the invention relates to a receiving module. The receiving module comprises means for performing at least the following steps: receiving a message regarding an event relevant to ventilation of a patient by a ventilator from a transmitting module, for example the transmitting module described above and below, via a data communication connection connecting the ventilator to at least one data processing device situated outside the ventilator for data communication purposes; generating a first control command for controlling a user interface so that information relevant to a user from the message is reproduced via the user interface, and/or generating a second control command for controlling an actuator of the ventilator using the message, wherein the actuator is designed to provide a respiratory gas pressure and/or a respiratory gas volumetric flow for ventilating the patient.

To put it another way, it is possible to evaluate the message for communication with the user, i.e., the patient or generally the operator of the ventilator, and/or for control of one or more ventilation functions of the ventilator.

As mentioned further above, the receiving module can be a hardware and/or software component of the ventilator and/or of the data processing device. It is conceivable, for example, for the data processing device to transmit the first and/or second control command via the data communication connection to the ventilator, where the respective control command is correspondingly processed and implemented. However, the first and/or second control command can also be generated in the ventilator.

The information relevant to the user, during reproduction via the user interface, can for example be displayed as text, image and/or video on a display and/or be output as speech and/or sounds via a loudspeaker.

It is possible for the second control command to be generated according to a current setting of at least one control parameter. The setting can be changed for example using reference values contained in the message, in order to improve the ventilation function.

A third aspect of the invention relates to a transmitting and receiving system comprising a transmitting module as described above and below and a receiving module as described above and below. The components of the transmitting and receiving system can be components of different apparatuses (networked with one another) and/or of one and the same apparatus. By way of example, the data processing device or the data processing devices can each comprise the receiving module, while the ventilator can comprise the transmitting module. However, the opposite case is also possible. Alternatively, both the ventilator and the data processing device(s) can each comprise a combination of a transmitting module and a receiving module (i.e., the transmitting and receiving system).

A fourth aspect of the invention relates to a ventilator. The ventilator comprises: a sensor for detecting at least one parameter which is relevant to ventilation of a patient by the ventilator; a data communication interface for connecting the ventilator to at least one data processing device situated outside the ventilator for data communication purposes; a user interface for a user to input information and/or for reproducing information relevant to a user; an actuator for providing a respiratory gas pressure and/or a respiratory gas volumetric flow for ventilating the patient; a transmitting module as described above and below and/or a receiving module as described above and below.

The actuator can be for example a fan with bellows drive and/or piston drive and/or in the form of a radial compressor.

The actuator can additionally be designed to control a respiratory gas composition, for example an oxygen, carbon dioxide and/or anaesthetic gas concentration in the respiratory gas.

A fifth aspect of the invention relates to a ventilation system. The ventilation system comprises a ventilator as described above and below and also a data processing device situated outside the ventilator (or a plurality of such data processing devices). The data processing device comprises a data communication interface for connecting the at least one data processing device to the ventilator for data communication purposes, a user interface for a user to input information and/or for reproducing information relevant to a user, and at least one of the modules described above and below for transmitting and/or receiving messages via the data communication interface.

From a geographical standpoint, the ventilator and the data processing device(s) can be separated from one another by several meters, several miles or even several hundred or several thousand meters or miles.

A sixth aspect of the invention relates to a computer program.

The computer program comprises a first set of instructions which, when the computer program is executed by a transmitting module, cause the transmitting module to perform at least the following steps: receiving input data, comprising sensor data and/or user data, wherein the sensor data define at least one parameter which is detected by at least one sensor and is relevant to ventilation of a patient by a ventilator, and the user data encode information regarding a patient who is ventilated or to be ventilated by a ventilator, said information being input by a user via a user interface; evaluating the input data in order to recognize an event from among a plurality of possible events, wherein each possible event is relevant to ventilation of the patient by the ventilator; transmitting a message regarding the recognized event to a receiving module, for example the receiving module described above and below, via a data communication connection connecting the ventilator to at least one data processing device situated outside the ventilator for data communication purposes.

In addition or as an alternative to the first set, the computer program comprises a second set of instructions which, when the computer program is executed by a receiving module, cause the receiving module to perform at least the following steps: receiving a message regarding an event relevant to ventilation of a patient by a ventilator from a transmitting module, for example the transmitting module described above and below, via a data communication connection connecting the ventilator to at least one data processing device situated outside the ventilator for data communication purposes; generating a first control command for controlling a user interface so that information relevant to a user from the message is reproduced via the user interface, and/or generating a second control command for controlling an actuator of the ventilator using the message, wherein the actuator is designed to provide a respiratory gas pressure and/or a respiratory gas volumetric flow for ventilating the patient.

“Computer program” can be understood to mean for example specific control and/or monitoring software and/or a specific app which can be executed on the ventilator and/or the data processing device (or the data processing devices).

A seventh aspect of the invention relates to a computer-readable medium on which the computer program is stored. The computer-readable medium can be a volatile or non-volatile data memory. By way of example, the computer-readable medium can be a hard disk, a USB storage device (universal serial bus), a RAM (random-access memory), a ROM (read-only memory), an EPROM (erasable programmable read-only memory), an EEPROM (electrically erasable programmable read-only memory), a flash memory or a combination of at least two of these examples. The computer-readable medium can also be a data communication network which enables program code to be downloaded (e.g. via the Internet), or a cloud.

It is pointed out that features of the transmitting module, of the receiving module and/or of the transmitting and receiving system as described above and below can also be features of the computer program and/or of the computer-readable medium (and vice versa).

An eighth aspect of the invention relates to a method for operating a transmitting module and/or a receiving module.

The method comprises at least the following steps for operating the transmitting module: receiving input data, comprising sensor data and/or user data, wherein the sensor data define at least one parameter which is detected by at least one sensor and is relevant to ventilation of a patient by a ventilator, and the user data encode information regarding a patient who is ventilated or to be ventilated by a ventilator, said information being input by a user via a user interface; evaluating the input data in order to recognize an event from among a plurality of possible events, wherein each possible event is relevant to ventilation of the patient by the ventilator; transmitting a message regarding the recognized event to a receiving module, for example the receiving module described above and below, via a data communication connection connecting the ventilator to at least one data processing device situated outside the ventilator for data communication purposes.

Additionally or alternatively, the method comprises at least the following steps for operating the receiving module: receiving a message regarding an event relevant to ventilation of a patient by a ventilator from a transmitting module, for example the transmitting module described above and below, via a data communication connection connecting the ventilator to at least one data processing device situated outside the ventilator for data communication purposes; generating a first control command for controlling a user interface so that information relevant to a user from the message is reproduced via the user interface, and/or generating a second control command for controlling an actuator of the ventilator using the message, wherein the actuator is designed to provide a respiratory gas pressure and/or a respiratory gas volumetric flow for ventilating the patient.

The method can be computer-implemented.

It is pointed out that features of the transmitting module, of the receiving module, of the transmitting and receiving system, of the computer program and/or of the computer-readable medium as described above and below can also be features of the method (and vice versa).

Various embodiments of the invention are described hereinafter. These embodiments should not be understood as restricting the scope of the invention.

In accordance with one embodiment, a first set of the input data can be received. In this case, the input data from the first set can be evaluated in order to recognize the event. If the event is recognized, then evaluating the input data can additionally comprise the following plausibilization steps: requesting at least one second set of the input data which deviates from the first set, and, if the second set is received, checking on the basis of the input data from the second set whether the recognized event is plausible, wherein a checking result is generated. The message can then be transmitted depending on the checking result.

In the simplest case, the checking result can be binary information such as “plausible” or “not plausible”. However, the checking result can also indicate a probability regarding the plausibility of the recognized event. The probability can then be compared with a threshold value, for example, in order to determine whether or not the recognized event is plausible.

Expediently, transmitting the message can be prevented if the checking result indicates that the recognized event is not plausible. The message is thus transmitted only if it is very probable that the recognized event is a real event. This firstly improves the data transfer efficiency compared with embodiments without such plausibilization, since overall fewer messages, namely only messages relating to plausible events, are transmitted. This secondly improves the recognition accuracy.

In accordance with one embodiment, the second set can define different parameters than the first set. To put it another way, for the plausibilization of the recognized event, it is possible to have recourse to a different type or different types of sensor data than for the (first) recognition of the event, for example to sensor data provided by an additional sensor which is situated outside the ventilator and/or carried by the patient. If, for the plausibilization, recourse is had to the same type or the same types of sensor data as for the recognition, then it may be the case that the sensor data are similarly erroneous in both cases, for instance on account of a defect or malfunction of the relevant sensor. Such errors can be avoided with the aid of this embodiment.

In accordance with one embodiment, evaluating the input data can comprise the following steps: converting at least one portion of the input data as classification data into class values by means of a classifier, wherein each class value indicates a probability and/or tendency for one of a plurality of event classes, wherein each event class corresponds to one of the possible events; recognizing the event using the class values.

“Class value” can be understood to mean for example a percentage between 0 and 1, a Boolean value such as “0” or “1”, or a tendency value from a continuous range of values.

The classifier can be for example a (naive) Bayes classifier, a k-nearest neighbors algorithm, a support vector machine, a clustering method, an artificial neural network (e.g. a multilayer perceptron, a convolutional neural network, a recurrent neural network, a long short-term memory) or a combination of at least two of these examples.

The recognized event can correspond for example to the event class with the largest or smallest class value.

By way of example, for evaluating the input data from the first set (see further above), it is possible to use a different, for example differently trained and/or differently structured, classifier than for evaluating the input data from the second set.

In accordance with one embodiment, the classifier can comprise an artificial neural network (see further above) which was trained with exemplary classification data in order to convert the classification data into the class values. The exemplary classification data can be training data in the form of recorded and correspondingly pre-processed sensor and/or user data. The network may have been trained in a supervised learning method, in which output values of the network are compared with known target values (also called labels), a discrepancy between the output values and the target values is determined with a loss function, and the loss function is minimized by iteratively adapting the weights of the network, for example in a (stochastic) gradient method. However, an unsupervised learning method is also possible. Such a classifier enables very accurate recognition. Furthermore, such a classifier can be updated by training with new training data with little outlay, for instance in order to enable new events to be recognized.

In accordance with one embodiment, a deviation of the at least one parameter from at least one target value can be determined. In this case, the event can be recognized depending on the deviation. To put it another way, it is possible to carry out one threshold value comparison or a plurality of threshold value comparisons for selecting a specific event from the set of possible events. These threshold or target values may be empirical values and/or may have been ascertained in experiments, for example.

In accordance with one embodiment, a priority can be assigned to each possible event and the message can be transmitted according to the priority of the recognized event. In this case, the priorities of different events can differ from one another. This makes it possible to treat important events with preference vis-à-vis less important events. To put it another way, messages relating to different recognized events can be transmitted with different priorities. By way of example, a message can be transmitted only if the priority of the event indicated by the message is greater than a specific threshold value. A preselection of messages to be transmitted can thus be made. This can further improve the efficiency of the data communication. Moreover, it is thereby possible to reduce the risk of data losses compared with embodiments in which all messages are sent with the same priority.

In accordance with one embodiment, a value for at least one data parameter can be determined depending on the recognized event, wherein the at least one data parameter defines the amount of data which are transmitted in the course of transmitting the message.

To put it another way, by setting the data parameter or the data parameters, it is possible to have a targeted influence on the loading of the data communication connection in the course of transmitting the message. In the course of transmitting the (same) message, more or fewer data can thus be sent, depending on what setting was chosen for the data parameter or data parameters. The data consumption can thus be controlled very accurately depending on the message content.

“Data parameter” can be understood to mean for example a data throughput in the course of transmitting the message, a maximum size of the message, a file format of the message, a parameter of a compression method for compressing the message, or a parameter of an encryption method for encrypting the message.

The value for the data parameter or data parameters can additionally be determined taking account of a current capacity utilization of the data communication connection and/or a priority assigned to the recognized event (see further above). In this respect, for example, in the course of transmitting a message relating to a higher-priority event, it is possible to transfer a larger amount of data than if the message relates to a lower-priority event. By way of example, the message can then comprise higher-resolution sensor data compared with when priority is lower. However, the opposite case is also possible, for instance in the event of high-capacity utilization of the data communication connection. It may be expedient here, under certain circumstances, to compress important messages to a greater degree than less-important messages, for instance in order to reduce the risk of data losses and/or to achieve the effect that the messages arrive at the respective receiver more rapidly.

Moreover, it may be expedient for messages having confidential contents (which may have a higher priority) to be sent with encryption, whereas messages having other, non-confidential contents (which may have a lower priority) can be sent without encryption.

If the message is transmitted in data packets, then it is expedient if, during the transmission of each data packet, a check is made to establish whether the data packet is received successfully. Each data packet which was not received successfully can then be transmitted at least one more time. It is thus possible to avoid data losses during the transmission of the message.

By way of example, a receiver of the data packets can be configured to transmit via the data communication connection a confirmation of reception for each data packet successfully received by the receiver. If the confirmation of reception is received within a specific waiting time since the last transmission of the data packet, then the data packet can be marked as successfully received. The next data packet can then be transmitted. By contrast, if the confirmation of reception is not received within the waiting time, then the data packet can be retransmitted, for example as often as until the confirmation of reception is received and/or a permissible number of transmission attempts is exceeded.

If the data packet cannot be successfully received even after retransmission, then a message to a user can be generated, for example, which advises the user of a possible data loss and/or comprises information for debugging.

In accordance with one embodiment, the possible events can comprise at least one of the following events:
a deviation of a ventilation frequency at which the patient is ventilated from a respiration frequency at which the patient respires lies outside a target range;
an inspiratory and/or expiratory pressure lies outside a target range;
a speed at which a pressure changes during transition between inspiration and expiration lies outside a target range;
a pressure loss during ventilation lies outside a target range;
the patient has respiratory distress during ventilation and/or as a reaction to the end of ventilation;
the ventilator hampers breathing out and/or breathing in;
the patient has sleeping problems (e.g. during ventilation);
the patient has a headache (e.g. during ventilation);
the patient's airways are too dry (e.g. during ventilation);
the patient has caught a cold;
an existing illness of the patient has significantly deteriorated or improved;
the patient has difficulties in operating the ventilator;
the compliance of the patient is insufficient.

In accordance with one embodiment, the message can comprise at least one of the following items of information:
at least one portion of the evaluated input data;
a description of the recognized event (such a description can for example also include a request to the patient to put on and/or to activate a specific additional sensor for detecting relevant sensor data);
a request to a user to input information regarding the recognized event via a user interface (such a request can include for example a list of questions directed to the patient);
a request to the patient to make contact with a contact person, in particular by way of a chat and/or video communication service;
an instruction as to how a specific complication in ventilation can be eliminated and/or prevented;
an encouragement that is intended to motivate the patient to better compliance;
a (reference) value—adapted to the recognized event—for at least one control parameter for controlling an actuator of the ventilator;
a patient identifier for uniquely identifying the patient;
an apparatus identifier for uniquely identifying the ventilator.

In accordance with one embodiment, the sensor data can define at least one of the following parameters regarding the patient: an inspiratory and/or expiratory pressure, an inspiratory and/or expiratory volumetric flow, a respiratory gas volume per breath, an inspiration and/or expiration time, a ventilation frequency, a respiration frequency, a respiratory gas composition, a respiration effort (also called effort), a respiration sound, a heart rate, a blood oxygen saturation, a blood pressure, a blood sugar level, a body temperature, a body weight, a body movement.

“Body movement” can be understood to mean for example a trunk, arm, leg or head movement or a walking movement.

In accordance with one embodiment, the sensor data can define a temporal profile of the at least one parameter, for example a respiratory pressure curve and/or respiratory flow curve. To put it another way, the sensor data may have been generated in different time periods. The event can thus not be recognized on the basis of a time series of measured values regarding the parameter or parameters, which improves the recognition accuracy compared with embodiments that do not take account of the temporal profile.

In accordance with one embodiment, the data communication interface of the ventilator can be configured to additionally connect the ventilator to at least one additional sensor for detecting at least one parameter which is relevant to the ventilation of the patient, for data communication purposes, said at least one additional sensor being situated outside the ventilator and/or carried by the patient.

The additional sensor can be configured for example for measuring a respiration effort, a respiration sound, a heart rate, a blood oxygen saturation, a blood pressure, a blood sugar level, a body temperature, a body weight, a body movement or a combination of at least two of these examples.

The additional sensor can be configured to transmit its sensor data directly to the data communication interface of the ventilator. Alternatively, the additional sensor can be configured to transmit its sensor data to the data communication interface via the detour of a mobile terminal such as, for example, a smartphone, a smartwatch or a fitness or sleep tracker. In this case, the additional sensor can be integrated into the mobile terminal, i.e. arranged in and/or on the housing thereof.

### BRIEF DESCRIPTION OF THE DRAWINGS

Embodiments of the invention are described hereinafter with reference to the accompanying drawings. Neither the description nor the drawings should be understood as restricting the scope of the invention.

FIG. 1 shows a ventilation system in accordance with one embodiment of the invention.

FIG. 2 shows components of a transmitting and receiving system in accordance with one embodiment of the invention.

The drawings are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

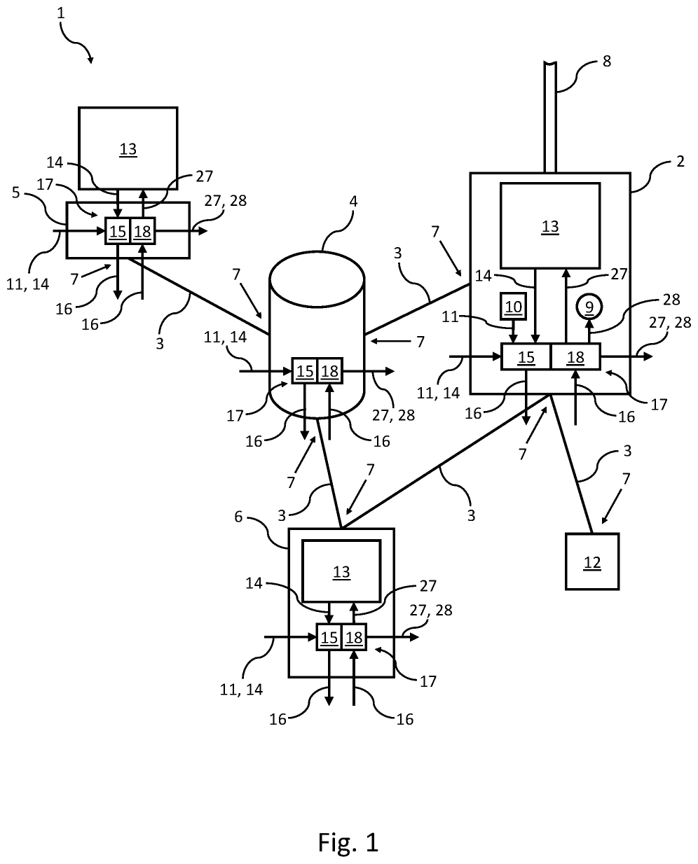

FIG. 1 shows a ventilation system 1, which networks a ventilator 2 via a data communication connection 3 to a plurality of data processing devices, here a first data processing device 4 in the form of a (cloud) server 4, a second data processing device 5 in the form of a PC 5 and a third data processing device 6 in the form of a smartphone 6, for data communication purposes, for example via the Internet, WLAN, Bluetooth and/or mobile radio. For this purpose, each of the apparatuses 2, 4, 5, 6 is equipped with a corresponding data communication interface 7.

In this example, the data communication connection 3 connects the ventilator 2 to the PC 5 and the smartphone 6 in each case via the server 4. However, a direct data communication connection 3 between the ventilator 2 and the PC 5 and/or, as shown here, between the ventilator 2 and the smartphone 6, is also possible, for example via Bluetooth.

The ventilator 2 comprises a ventilation tube 8 for providing a respiratory gas with which a patient can be ventilated invasively or noninvasively. The respiratory gas can be controllable in terms of its pressure and/or volumetric flow (flow) by means of a suitable actuator 9 of the ventilator 2.

Moreover, the ventilator 2 comprises a sensor 10 for detecting parameters relevant to ventilation, i.e. to control of the respiratory pressure and/or flow, and for providing corresponding sensor data 11. The sensor data 11 can encode for example measurement series for each parameter, i.e. the temporal profile thereof.

A portion of the sensor data 11 can optionally be provided by an additional, external sensor 12, which can be connected to the ventilator 2 in a wired and/or wireless manner, preferably via Bluetooth and/or WLAN, for data communication purposes. The additional sensor 12 can be configured to communicate directly with the ventilator 2 via a dedicated data communication interface 7 independently of other network subscribers. However, indirect data communication between the additional sensor 12 and the ventilator 2 is also possible, here for example via the smartphone 6. The additional sensor 12 can be portable for carrying by the patient, or stationary.

Examples of the parameters mentioned above are an inspiratory and/or expiratory pressure, an inspiratory and/or expiratory volumetric flow, a respiratory gas volume per breath, an inspiration and/or expiration time, a ventilation frequency, a respiration frequency, a respiratory gas composition, a respiration effort or a respiration sound, a heart rate, a blood oxygen saturation, a blood pressure, a blood sugar level, a body temperature, a body weight, or a body movement.

By way of example, the additional sensor 12 can be configured to detect parameters such as the respiration frequency, the respiration effort, the respiration sound, the heart rate, the blood oxygen saturation, the blood pressure, the blood sugar level, the body temperature, the body weight or the body movement.

Furthermore, the ventilator 2 comprises a user interface 13, here in the form of a touch display, via which a user (i.e. the patient or an operator of the ventilator 2) can input information, in particular regarding ventilation (e.g. operating commands or indications for use), and which can display information to the user, in particular regarding ventilation (e.g. chosen settings or measured values in diagrammatic form).

The sensor data 11 and/or user data 14 encoding the information input via the user interface 13 are received as input data in a transmitting module 15 of the ventilator 2, are evaluated there and are converted into a message 16 depending on the evaluation result, which message is then transmitted to one or more other network subscribers via the data communication connection 3.

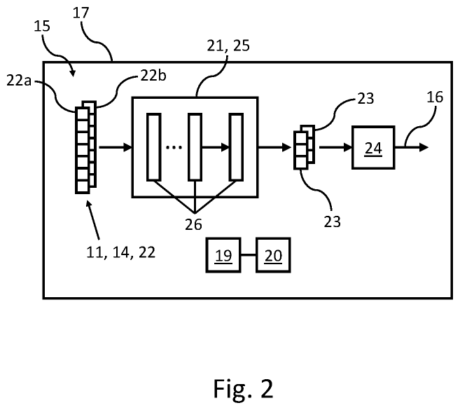

The transmitting module 15 (and all modules described below) can be implemented as hardware and/or software. In this example, the transmitting module 15 is part of a transmitting and receiving system 17 of the ventilator 2, referred to below as control system 17 for short (see also FIG. 2), which comprises a receiving module 18 for receiving a message 16 besides the transmitting module 15.

As shown in FIG. 2, the control system 17 comprises a memory 19 with a specific computer program stored therein, and a processor 20 configured to execute instructions of the computer program, whereby the following method is performed.

After the reception of the input data in the transmitting module 15, the input data are evaluated in order to select a specific event from a plurality of possible events. Such a possible event can be for example a medical and/or technical complication during the ventilation of the patient.

For this purpose, the transmitting module 15 can implement a classifier 21, which calculates a plurality of class values 23 from the input data (or a portion thereof) as classification data 22, wherein each class value 23 is linked with one out of a plurality of predefined event classes and indicates a probability or tendency for the respective event class. The class values 23 can then be input into an evaluation unit 24, which selects one of the event classes on the basis of the class values 23 and determines the selected event class, more precisely the (possible) event assigned to the selected event class, as a recognized event.

The evaluation unit 24 thereupon generates a message 16 regarding the recognized event and transmits this message via the data communication connection 3 to one or more of the other network subscribers, here to the server 4 and/or the smartphone 6. The PC 5 can then retrieve the message 16 from the server 4 (in this case, the user interface 13 of the PC 5 can function as a user interface for the server 4).

As shown in FIG. 2, the classifier 21 can comprise an artificial neural network 25 (referred to below as network 25 for short) having a plurality of trainable intermediate layers 26. The network 25 may have been trained with exemplary classification data, for example recorded sensor and/or user data, and corresponding target values in order to convert the classification data 22 into the class values 23. The training may have been carried out online, i.e., in the (operational) ventilator 2, and/or offline, i.e. outside the ventilator 2.

Prior to input into the network 25, the classification data 22 can be pre-processed in order to obtain an input vector composed of input values that is suitable for the processing by the network 25. In addition, the network 25 can be configured to provide the class values 23 in the form of an output vector that is suitable for the further processing in the evaluation unit 24.

In this example, the network 25 processes two sets 22a, 22b of the classification data 22 (in parallel and/or sequentially), wherein the network 25 generates an output vector having class values 23 for each of the sets 22a, 22b. The sets 22a, 22b can differ from one another for example in terms of their data type. By way of example, a first set 22a can comprise a different type of sensor data 11 and/or a different type of user data 14 than a second set 22b. The evaluation unit 24 then evaluates both output vectors in order to recognize the event and to plausibilize it.

The network 25 can also process more than two, for example more than ten, more than a hundred or more than a thousand, sets of the classification data 22 (in parallel and/or sequentially) in order to recognize the event and to plausibilize it.

Additionally or alternatively, the event can be recognized on the basis of one or more threshold value comparisons.

It is possible for different recognized events to be of differing importance for the ventilation of the patient. Accordingly, a message 16 relating to an important event can be transmitted with higher priority than a message 16 relating to a less important event.

For this purpose, for example, each of the event classes mentioned above can be assigned a specific transmission priority that is taken into account in the course of transmitting the respective message 16.

Additionally or alternatively, for each message 16, specific data parameters that influence the amount of data that arises in the course of transmitting the message 16 can be set individually depending on the content of the message 16, i.e. depending on the recognized event.

The message 16 can then be received by a receiving module 18 in one or more of the data processing devices 4, 5, 6, evaluated there and converted into one or more control commands, more precisely into a first control command 27 for controlling a user interface 13 of the respective data processing device and/or into a second control command 28 for controlling the actuator 9. For this purpose, the receiving module 18 can transmit one, or both, of the control commands 27, 28 via the data communication connection 3 to the ventilator 2 and/or to at least one of the other network subscribers. For example, the respective network subscriber can then control its user interface 13 according to the first control command 27 such that specific contents from the message 16 are reproduced for the respective user.

It is possible that at least one or, as here, each of the data processing devices 4, 5, 6 is equipped with a transmitting and receiving system 17, i.e. can receive and transmit messages 16. In this case, the receiving module 18 of the ventilator 2 can be configured to receive the messages 16 from the respective data processing device and, as described above, to convert them into first control commands 25 and/or second control commands 26 (and optionally to forward these via the data communication connection 3).

It is also possible in this case that at least one or, as here, each of the network subscribers receives, via the data communication connection 3, at least one portion of the user data 14 output by a user interface 13 of at least one other network subscriber and/or at least one portion of the sensor data 11 output by at least one of the sensors 10, 12 and uses said portion(s) as the input data.

Events that can be recognized by the transmitting module 15 in the case of ventilation not being set optimally and/or in the case of a change in the ventilation requirement can be, for example:
the ventilator 2 and the patient are not synchronous;
the pressure assistance afforded by the ventilator 2 is too great or too little;
the splinting pressure generated by the ventilator 2 (i.e. the expiratory pressure) is too low;
the ventilator 2 obstructs breathing out by the patient;
the pressure ramps between expiratory and inspiratory pressures at the beginning or at the end of inspiration are unphysiological, i.e. the pressure changes too rapidly or too slowly;
the patient has respiratory distress during ventilation or after the ventilation function has been switched off;
the patient's ventilation requirement has changed significantly (e.g. on account of physical or sporting activity, on account of an infection, when getting up, when going to bed).

Events that can be recognized by the transmitting module 15 when ventilation is applied to the airways can be, for example:
the patient interface is not airtight and/or the patient is breathing with their mouth open, and so sufficient pressure cannot be applied to the lower airways;
drying out of the airways;
insufficient motivation on the part of the patient;
problems when operating the ventilator 2;
the equipment of the ventilator 2 disturbs the patient's sleep;
the patient has a headache;
the patient has sinusitis.

These events can be recognized in particular by way of:
evaluating the respiratory flow curve on its own or in combination with the respiratory pressure curve (this can be done for example by sending sensor data 11 with particularly high resolution in a message 16);
evaluating the respiratory effort, which can be calculated from the respiratory flow curve and the respiratory pressure curve and/or can be measured by means of an effort sensor;
evaluating information input by the patient via a user interface 13;
applying one or more additional sensors 12 and evaluating the respective sensor data 11 owing to an initial suspicion that arose during the first evaluation of the input data.

In such cases, the message 16 can comprise for example one or more of the following items of information:
high-resolution sections of the sensor data 11, for example for visual evaluation by an expert who can then make a decision about a suitable intervention;
a proposal for an own intervention that can be carried out by the patient themselves;
settings for automatically optimizing the ventilation function;
an instruction for optimizing or for exchanging ventilation accessories such as e.g. the ventilation tube 8 or a ventilation mask;
a motivating message;
a request to the patient to make contact with a contact person;
an invitation to a consultation for the patient via a specific app by way of chat and/or video telephony.

Furthermore, upon recognition of an acute deterioration in an existing illness of the patient, for example appertaining to the heart, lungs or brainstem, the message 16 can comprise information specifically directed to a medical carer for further interventions in addition to ventilation.

Various embodiments of the invention are described again below using different wording.

The ventilator 2 can be configured to ascertain and store sensor data 11 detected by at least one sensor 10, 12, in particular flow and pressure curves, or signals derived therefrom. Such signals can indicate for example at least one of the following items of information: leakage, respiration frequency, tidal volume, respiration events, synchronism indicators, sleep stage indicators, cough indicators, secretion formation indicators, ambient conditions (e.g., temperature, air humidity, air pressure).

Additionally or alternatively, data from optional external sensors can be ascertained and stored. These data can indicate for example at least one of the following items of information: carbon dioxide content of the exhaled air, blood or surroundings, oxygen content of the inhaled air, blood or surroundings, body temperature, ECG signals, movement signals (e.g. generated by acceleration, radar, ultrasonic, distance or pressure sensors, for example in bed, or by electromagnetic fields), information about sequences such as the start or end of therapy, configuration data, transmission processes, technical faults, results of self-tests, wear data, filling levels of a water or oxygen reservoir.

These sensor data are defined and evaluated as input data in order to recognize an event from among a plurality of possible events, wherein each possible event is relevant to ventilation of the patient by the ventilator 2.

If an event is recognized, then the ventilator is caused to read out from the memory sensor data that were stored in a specific time period, and to transmit them with a specific resolution, defined for example depending on the respective type of event, via a data communication connection 3 to a receiving module 18, wherein the data communication connection 3 connects the ventilator 2 to at least one data processing device 4, 5, 6 situated outside the ventilator 2 for data communication purposes.

The time period can be predefined per event. By way of example, the time period can comprise a fixed first time period before and/or a fixed second time period after the point in time of the event (in the case of coughing e.g., two minutes before that and/or after that). The time period can also be defined depending on the last transmission process. Alternatively, the data since the last transmission process can be sent as soon as the event occurs.

Since the time period at the point in time when the event is recognized has possibly not finished yet, the transmission process can be initiated in a delayed manner in some cases. If, for example in the case of coughing as recognized event, data regarding the event itself and regarding a specific time period before and after the event (e.g., 2 minutes in each case) are intended to be transferred with high resolution, it is possible, at the point in time when the event is recognized, to start a timer for 2 minutes, after the expiry of which finally 4 minutes of high-resolution data are transferred.

As an alternative to immediate sending after the time period has elapsed, the data can firstly be stored temporarily in a transmission buffer, i.e., earmarked for sending. When the next transmission event then occurs, for example on a regular cycle several times per day after a request for transmission by the user or the data processing device, all entries in the transmission buffer are transferred. By way of example, whenever the ventilator recognizes a pause in breathing, 30, 60, 90 or 180 (or more) seconds of high-resolution data before and after this event are stored temporarily in the transmission buffer. The entries are transmitted jointly during the next transfer. Alternatively, for each type of event, a previously defined number of examples, for example the first 5, 10, 20 or 100 examples, are stored temporarily in the transmission buffer as representatives in order that the data processing device and/or a human evaluator can use the examples to analyze what exactly is happening during ventilation.

Optionally, the long-term memory for the (high-resolution) data in the apparatus can be erased on a rolling basis, for example after a specific number of hours or days. In this case, the storage duration can be equal to the maximum length of the time period to be sent over all defined types of event.

It is possible for the transmission process to be initiated, in the case of specific events, not as early as upon the first occurrence but rather only after repeated occurrence, i.e. when a specific frequency is reached (e.g. 2 times per minute, 10 times per hour or 5 times per day).

Examples of technical events: expiry of the timer for data transfer; request for data by the receiving module of a counterpart station; technical alarm or fault; reaching a specific number of alarms/faults; transmission error regarding a specific transmission path (hence triggering of a different transmission path); starting or stopping of therapy; alteration of the therapy settings; excessively long or excessively short pause in therapy; impermissible ambient conditions; blockage of ventilation tube, patient interface and/or apparatus inlet; excessively high leakage.

Manual events can be input by the user via the user interface, for example by the patient themselves or some other person who is in proximity to the patient or else not in proximity to the patient, such as a caregiver, an expert or a relative. The initiation can be effected via the user interface for example directly on the apparatus and/or via a remote control, a network command, an app, a cloud application or an SMS.

Examples of medical events: pauses in breathing, coughs, changes in the carbon dioxide or oxygen content, heart arrhythmia, stopping of the heartbeat, disconnection of the ventilation, disconnection of an additional sensor, change in body temperature, signaling of a complication or worsening of a symptom of the patient via a human-machine interface on the ventilator or on some other module (e.g. a mobile terminal), increased amount of secretion in the airways, undershooting of a breath volume or respiration time volume.

The events can be recognized individually or in combination.

Finally, it is pointed out that terms such as “have”, “comprise”, “include”, “with”, etc., do not exclude other elements or steps, and indefinite articles such as “a” or “an” do not exclude a plurality.

Furthermore, it is pointed out that features or steps described with reference to one of the embodiments above can also be used in combination with features or steps described with reference to other embodiments from among the embodiments above.

Reference signs in the claims should not be understood as restricting the scope of the subject matter defined by the claims.

To sum up, the present invention provides:
1. A transmitting module, which module comprises elements configured for:
   receiving input data which comprise: sensor data defining at least one parameter which is detected by at least one sensor and is relevant to ventilation of a patient by a ventilator, and/or user data encoding information regarding a patient who is ventilated or to be ventilated by a ventilator, said information being input by a user via a user interface;
   evaluating the input data in order to recognize an event from among a plurality of possible events, each possible event being relevant to ventilation of the patient by the ventilator;
   transmitting a message regarding the recognized event to a receiving module via a data communication connection connecting the ventilator to at least one data processing device situated outside the ventilator for data communication purposes.
2. The transmitting module of item 1, wherein
   a first set of the input data is received and the input data from the first set are evaluated in order to recognize the event;
   evaluating the input data additionally comprises, if the event is recognized:
   requesting at least one second set of the input data which deviates from the first set;
   if the second set is received: checking on the basis of the input data from the second set whether the recognized event is plausible, wherein a checking result is generated; and
   the message is transmitted depending on the checking result.
3. The transmitting module of any of the preceding items,
   wherein evaluating the input data comprises:
   converting at least one portion of the input data as classification data into class values by means of a classifier, each class value indicating a probability and/or tendency for one of a plurality of event classes, wherein each event class corresponds to one of the possible events;
   recognizing the event using the class values.
4. The transmitting module of item 3,
   wherein the classifier comprises an artificial neural network which was trained with exemplary classification data in order to convert the classification data into the class values.
5. The transmitting module of any of the preceding items, wherein a deviation of the at least one parameter from at least one target value is determined and the event is recognized depending on the deviation.
6. The transmitting module of any of the preceding items, wherein a priority is assigned to each possible event and the message is transmitted according to the priority of the recognized event.
7. The transmitting module of any of the preceding items, wherein a value for at least one data parameter is determined depending on the recognized event, the at least one data parameter defining the amount of data which are transmitted in the course of transmitting the message.
8. The transmitting module of any of the preceding items, wherein the possible events comprise at least one of the following events:
   a deviation of a ventilation frequency at which the patient is ventilated from a respiration frequency at which the patient respires lies outside a target range;
   an inspiratory and/or expiratory pressure lies outside a target range;
   a speed at which a pressure changes during transition between inspiration and expiration lies outside a target range;
   a pressure loss during ventilation lies outside a target range;
   the patient has respiratory distress during ventilation and/or as a reaction to the end of ventilation;
   the ventilator hampers breathing out and/or breathing in;
   the patient has sleeping problems;
   the patient has a headache;
   the patient's airways are too dry;
   the patient has caught a cold;
   an existing illness of the patient has significantly deteriorated or improved;
   the patient has difficulties in operating the ventilator;
   a compliance of the patient is insufficient.
9. The transmitting module of any of the preceding items, wherein the message comprises at least one of the following items of information:
   at least one portion of the evaluated input data;
   a description of the recognized event;
   a request to a user to input information regarding the recognized event via a user interface;
   a request to the patient to make contact with a contact person, in particular by way of a chat and/or video communication service;
   an instruction as to how a specific complication in ventilation can be eliminated and/or prevented;
   an encouragement that is intended to motivate the patient to better compliance;
   a value—adapted to the recognized event—for at least one control parameter for controlling an actuator of the ventilator;
   a patient identifier for uniquely identifying the patient;
   an apparatus identifier for uniquely identifying the ventilator.
10. A receiving module, the module comprising elements configured for:
   receiving a message regarding an event relevant to ventilation of a patient by a ventilator from a transmitting module via a data communication connection connecting the ventilator to at least one data processing device situated outside the ventilator for data communication purposes;
   generating a first control command for controlling a user interface so that information relevant to a user from the message is reproduced via the user interface; and/or
   generating a second control command for controlling an actuator of the ventilator using the message, the actuator being designed to provide a respiratory gas pressure and/or a respiratory gas volumetric flow for ventilating the patient.
11. A transmitting and receiving system, wherein the system comprises:
   a transmitting module according to any of items 1 to 9;
   a receiving module according to item 10.
12. A ventilator, wherein the ventilator comprises:
   a sensor for detecting at least one parameter which is relevant to ventilation of a patient by the ventilator;
   a data communication interface for connecting the ventilator to at least one data processing device situated outside the ventilator for data communication purposes;
   a user interface for a user to input information and/or for reproducing information relevant to a user;
   an actuator for providing a respiratory gas pressure and/or a respiratory gas volumetric flow for ventilating the patient;
   a transmitting module according to any of items 1 to 9 and/or a receiving module according to item 10.
13. The ventilator of item 12,
   wherein the data communication interface is configured to additionally connect the ventilator to at least one additional sensor for detecting at least one parameter which is relevant to the ventilation of the patient, for data communication purposes, said at least one additional sensor being situated outside the ventilator and/or carried by the patient.
14. A ventilation system, wherein the system comprises:
   a ventilator according to any of items 12 and 13;
   a data processing device situated outside the ventilator and comprising:
   a data communication interface for connecting the data processing device to the ventilator for data communication purposes,
   a user interface for a user to input information and/or for reproducing information relevant to a user,
   a transmitting module according to any of items 1 to 9 and/or a receiving module according to item 10.
15. A computer program, wherein the program comprises a first set of instructions which, when the computer program is executed by a transmitting module, cause the transmitting module to:
   receive input data, comprising: sensor data defining at least one parameter which is detected by at least one sensor and is relevant to ventilation of a patient by a ventilator, and/or user data encoding information regarding a patient who is ventilated or to be ventilated by a ventilator, said information being input by a user via a user interface;
   evaluate the input data in order to recognize an event from among a plurality of possible events, wherein each possible event is relevant to ventilation of the patient by the ventilator;
   transmit a message regarding the recognized event to a receiving module via a data communication connection connecting the ventilator to at least one data processing device situated outside the ventilator for data communication purposes;
   and/or comprising a second set of instructions which, when the computer program is executed by a receiving module, cause the receiving module to:
   receive a message regarding an event relevant to ventilation of a patient by a ventilator from a transmitting module via a data communication connection connecting the ventilator to at least one data processing device situated outside the ventilator for data communication purposes;
   generate a first control command for controlling a user interface so that information relevant to a user from the message is reproduced via the user interface; and/or
   generate a second control command for controlling an actuator of the ventilator using the message, wherein the actuator is designed to provide a respiratory gas pressure and/or a respiratory gas volumetric flow for ventilating the patient.
16. A computer-readable medium on which the computer program of item 15 is stored.
17. A method for operating a transmitting module and/or a receiving module, wherein the method for operating the transmitting module comprises:
   receiving input data, comprising: sensor data defining at least one parameter which is detected by at least one sensor and is relevant to ventilation of a patient by a ventilator, and/or user data encoding information regarding a patient who is ventilated or to be ventilated by a ventilator, said information being input by a user via a user interface;
   evaluating the input data in order to recognize an event from among a plurality of possible events, wherein each possible event is relevant to ventilation of the patient by the ventilator;
   transmitting a message regarding the recognized event to a receiving module via a data communication connection connecting the ventilator to at least one data processing device situated outside the ventilator for data communication purposes;
   and/or wherein the method for operating the receiving module comprises:
   receiving a message regarding an event relevant to ventilation of a patient by a ventilator from a transmitting module via a data communication connection connecting the ventilator to at least one data processing device situated outside the ventilator for data communication purposes;
   generating a first control command for controlling a user interface so that information relevant to a user from the message is reproduced via the user interface; and/or
   generating a second control command for controlling an actuator of the ventilator using the message, wherein the actuator is designed to provide a respiratory gas pressure and/or a respiratory gas volumetric flow for ventilating the patient.

### LIST OF REFERENCE SIGNS

1 Ventilation system
2 Ventilator
3 Data communication connection
4 First data processing device/server
5 Second data processing device/PC
6 Third data processing device/smartphone
7 Data communication interface
8 Ventilation tube
9 Actuator
10 Sensor
11 Sensor data
12 Additional sensor
13 User interface
14 User data
15 Transmitting module
16 Message
17 Transmitting and receiving system/control system
18 Receiving module
19 Memory
20 Processor
21 Classifier
22 Classification data
22a First set
22b Second set
23 Class value
24 Evaluation unit
25 Artificial neural network
26 Intermediate layer
27 First control command
28 Second control command

What is claimed is:

1. A transmitting module, wherein the module comprises elements configured for:
receiving input data which comprise: sensor data defining at least one parameter which is detected by at least one sensor and is relevant to ventilation of a patient by a ventilator, and/or user data encoding information regarding a patient who is ventilated or to be ventilated by a ventilator, said information being input by a user via a user interface;
evaluating the input data in order to recognize an event from among a plurality of possible events, wherein each possible event is relevant to ventilation of the patient by the ventilator;
transmitting a message regarding the recognized event to a receiving module via a data communication connection connecting the ventilator to at least one data processing device situated outside the ventilator for data communication purposes.

2. The transmitting module of claim 1, wherein
a first set of the input data is received and the input data from the first set are evaluated in order to recognize the event;
evaluating the input data additionally comprises, if the event is recognized:
requesting at least one second set of the input data which deviates from the first set;
if the second set is received: checking on the basis of the input data from the second set whether the recognized event is plausible, wherein a checking result is generated; and
the message is transmitted depending on the checking result.

3. The transmitting module of claim 1,
wherein evaluating the input data comprises:
converting at least one portion of the input data as classification data into class values by means of a classifier, each class value indicating a probability and/or tendency for one of a plurality of event classes, wherein each event class corresponds to one of the possible events;
recognizing the event using the class values.

4. The transmitting module of claim 3, wherein the classifier comprises an artificial neural network which was trained with exemplary classification data in order to convert the classification data into the class values.

5. The transmitting module of claim 1, wherein a deviation of the at least one parameter from at least one target value is determined and the event is recognized depending on the deviation.

6. The transmitting module of claim 1, wherein a priority is assigned to each possible event and the message is transmitted according to the priority of the recognized event.

7. The transmitting module of claim 1, wherein a value for at least one data parameter is determined depending on the recognized event, the at least one data parameter defining the amount of data which are transmitted in the course of transmitting the message.

8. The transmitting module of claim 1, wherein the possible events comprise at least one of the following events:
a deviation of a ventilation frequency at which the patient is ventilated from a respiration frequency at which the patient respires lies outside a target range;
an inspiratory and/or expiratory pressure lies outside a target range;
a speed at which a pressure changes during transition between inspiration and expiration lies outside a target range;
a pressure loss during ventilation lies outside a target range;
the patient has respiratory distress during ventilation and/or as a reaction to the end of ventilation;
the ventilator hampers breathing out and/or breathing in;
the patient has sleeping problems;
the patient has a headache;
the patient's airways are too dry;
the patient has caught a cold;
an existing illness of the patient has significantly deteriorated or improved;
the patient has difficulties in operating the ventilator;
a compliance of the patient is insufficient.

9. The transmitting module of claim 1, wherein the message comprises at least one of the following items of information:
at least one portion of the evaluated input data;
a description of the recognized event;
a request to a user to input information regarding the recognized event via a user interface;
a request to the patient to make contact with a contact person, in particular by way of a chat and/or video communication service;
an instruction as to how a specific complication in ventilation can be eliminated and/or prevented;
an encouragement that is intended to motivate the patient to better compliance;
a value—adapted to the recognized event—for at least one control parameter for controlling an actuator of the ventilator;
a patient identifier for uniquely identifying the patient;
an apparatus identifier for uniquely identifying the ventilator.

10. A receiving module, wherein the module comprises elements configured for:
receiving a message regarding an event relevant to ventilation of a patient by a ventilator from a transmitting module via a data communication connection connecting the ventilator to at least one data processing device situated outside the ventilator for data communication purposes;
generating a first control command for controlling a user interface so that information relevant to a user from the message is reproduced via the user interface; and/or
generating a second control command for controlling an actuator of the ventilator using the message, the actuator being designed to provide a respiratory gas pressure and/or a respiratory gas volumetric flow for ventilating the patient.

11. A transmitting and receiving system, wherein the system comprises:
the transmitting module of claim 1;
a receiving module which comprises elements configured for:
receiving a message regarding an event relevant to ventilation of a patient by a ventilator from a transmitting module via a data communication connection connecting the ventilator to at least one data processing device situated outside the ventilator for data communication purposes;
generating a first control command for controlling a user interface so that information relevant to a user from the message is reproduced via the user interface; and/or
generating a second control command for controlling an actuator of the ventilator using the message, the actuator being designed to provide a respiratory gas pressure and/or a respiratory gas volumetric flow for ventilating the patient.

12. A ventilator, wherein the ventilator comprises:
a sensor for detecting at least one parameter which is relevant to ventilation of a patient by the ventilator;
a data communication interface for connecting the ventilator to at least one data processing device situated outside the ventilator for data communication purposes;
a user interface for a user to input information and/or for reproducing information relevant to a user;
an actuator for providing a respiratory gas pressure and/or a respiratory gas volumetric flow for ventilating the patient;
a transmitting module according to claim 1 and/or a receiving module which comprises elements configured for:
receiving a message regarding an event relevant to ventilation of a patient by a ventilator from a transmitting module via a data communication connection connecting the ventilator to at least one data processing device situated outside the ventilator for data communication purposes;
generating a first control command for controlling a user interface so that information relevant to a user from the message is reproduced via the user interface; and/or
generating a second control command for controlling an actuator of the ventilator using the message, the actuator being designed to provide a respiratory gas pressure and/or a respiratory gas volumetric flow for ventilating the patient.

13. The ventilator of claim 12, wherein the data communication interface is configured to additionally connect the ventilator to at least one additional sensor for detecting at least one parameter which is relevant to the ventilation of the patient, for data communication purposes, said at least one additional sensor being situated outside the ventilator and/or carried by the patient.

14. A ventilation system, wherein the system comprises:
a ventilator according to claim 12;
a data processing device situated outside the ventilator and comprising:
a data communication interface for connecting the data processing device to the ventilator for data communication purposes,
a user interface for a user to input information and/or for reproducing information relevant to a user,
a transmitting module which comprises elements configured for:
receiving input data which comprise: sensor data defining at least one parameter which is detected by at least one sensor and is relevant to ventilation of a patient by a ventilator, and/or user data encoding information regarding a patient who is ventilated or to be ventilated by a ventilator, said information being input by a user via a user interface;
evaluating the input data in order to recognize an event from among a plurality of possible events, wherein each possible event is relevant to ventilation of the patient by the ventilator;
transmitting a message regarding the recognized event to a receiving module via a data communication connection connecting the ventilator to at least one data processing device situated outside the ventilator for data communication purposes;
and/or a receiving module which comprises elements configured for:
receiving a message regarding an event relevant to ventilation of a patient by a ventilator from a transmitting module via a data communication connection connecting the ventilator to at least one data processing device situated outside the ventilator for data communication purposes;
generating a first control command for controlling a user interface so that information relevant to a user from the message is reproduced via the user interface; and/or
generating a second control command for controlling an actuator of the ventilator using the message, the actuator being designed to provide a respiratory gas pressure and/or a respiratory gas volumetric flow for ventilating the patient.

15. A computer program, wherein the program comprises a first set of instructions which, when the computer program is executed by a transmitting module, cause the transmitting module to:
receive input data, comprising: sensor data defining at least one parameter which is detected by at least one sensor and is relevant to ventilation of a patient by a ventilator, and/or user data encoding information regarding a patient who is ventilated or to be ventilated by a ventilator, said information being input by a user via a user interface;
evaluate the input data in order to recognize an event from among a plurality of possible events, wherein each possible event is relevant to ventilation of the patient by the ventilator;
transmit a message regarding the recognized event to a receiving module via a data communication connection connecting the ventilator to at least one data processing device situated outside the ventilator for data communication purposes;
and/or comprising a second set of instructions which, when the computer program is executed by a receiving module, cause the receiving module to:
receive a message regarding an event relevant to ventilation of a patient by a ventilator from a transmitting module via a data communication connection connecting the ventilator to at least one data processing device situated outside the ventilator for data communication purposes;
generate a first control command for controlling a user interface so that information relevant to a user from the message is reproduced via the user interface; and/or
generate a second control command for controlling an actuator of the ventilator using the message, wherein the actuator is designed to provide a respiratory gas pressure and/or a respiratory gas volumetric flow for ventilating the patient.

16. A computer-readable medium on which the computer program of claim 15 is stored.
